(19) United States
(12) Patent Application Publication
(10) Pub. No.: US 2010/0116655 A1
(43) Pub. Date: May 13, 2010

(54) CHEMICAL MESSENGER SENSOR

(75) Inventors: Claire HARLEY, Kildare (IE); John COLLERAN, Kildare (IE); Bernadette ALCOCK, Kildare (IE); Carmel BRESLIN, Kildare (IE)

Correspondence Address: SCULLY SCOTT MURPHY & PRESSER, PC, 400 GARDEN CITY PLAZA, SUITE 300, GARDEN CITY, NY, 11530, US

(73) Assignee: NATIONAL UNIVERSITY OF IRELAND, MAYNOOTH, Co. Kildare (IE)

(21) Appl. No.: 12/267,996

(22) Filed: Nov. 10, 2008

(51) Int. Cl. G01N 33/74; C07D 495/04; B05D 3/14; G01N 33/50

(52) U.S. Cl. 204/403.01; 549/50; 427/2.13

(57) ABSTRACT

A sensor for the detection of chemical messengers is described herein. In particular a sensor for the detection of catecholamines, for example dopamine, epinephrine or norepinephrine, is reported. Catecholamines play pivotal roles as neurotransmitters and hormones in the human body. An electrode for detecting a catecholamine comprising a conducting or semi-conducting substrate, and a polymer comprising polyethylenedioxythiophene on said substrate is disclosed. The polymer is doped with a cyclodextrin macrocycle. Suitable cyclodextrin macrocycles include anionic cyclodextrin macrocycles, for example sulfonated β-cyclodextrins (CDs). Also, disclosed in sensor capable of selectively detecting a catecholamine in the presence of ascorbic acid (ascorbate).

### FIELD OF THE INVENTION

A sensor for the detection of chemical messengers is described herein. In particular a sensor for the detection of catecholamines, for example dopamine, epinephrine or norepinephrine, is reported. Methods of constructing sensors according to the present invention are also described. Suitable materials for the construction of such sensors are disclosed with a view to developing a sensor capable of real-time in-vivo catecholamine monitoring.

### BACKGROUND TO THE INVENTION

Catecholamines play pivotal roles as neurotransmitters and hormones in the human body. Of the many catecholamines, the most important in regulating human physiology are dopamine (DA), epinephrine (EP) and norepinephrine (norEP).

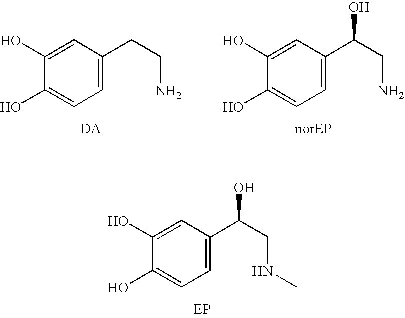

Dopamine is one of the most important chemical messengers/neurotransmitters in the human body. Abnormalities in dopamine concentrations have been linked to Parkinson's disease, Schizophrenia and Attention Deficit Hyperactivity Disorders. Furthermore, dopamine also plays a central role in drug addiction, depression and sleep regulation. Epinephrine and norepinephrine function as neurotransmitters in the brain and as hormones in blood circulation, and are the primary ligands for the adrenergic receptors of the sympathetic nervous system, responsible for regulating many physiological conditions such as heart rate and blood pressure.

Thus, the ability to reliably monitor dopamine, epinephrine and norepinephrine concentrations in the living brain could have far-reaching applications in the treatment of several mental disorders and the understanding of catecholamine function in many pathophysiological conditions.

The oxidation of catecholamines invariably proceeds via an o-Quinone intermediate. Oxidation of dopamine to dopamine-o-Quinone, followed by cyclisation and further oxidation is shown in Scheme 1. The mechanism is defined as ECE in that it involves an electrochemical step, followed by a chemical step and terminates in a further electrochemical step. A similar ECE mechanism applies to epinephrine and norepinephrine. Electrochemical detection is a most promising approach for the detection of catehcolamines in-vivo.

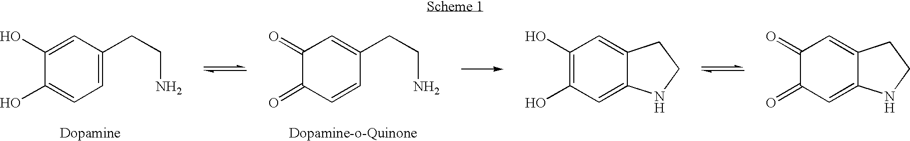

However, monitoring the concentration of species such as dopamine, epinephrine and norepinephrine is particularly challenging, because it co-exists with other interfering species. In particular, ascorbic acid (3) oxidises at the same potential as dopamine at bare electrodes. Other common interferants such as uric acid (4), DOPAC (1) and homovanillic acid (2) are also problematic. The exclusion of these last two species is particularly advantageous as these are metabolites of dopamine, which are known to poison other biosensors reducing their sensitivity.

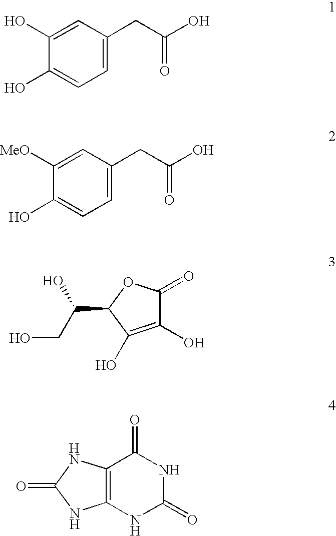

The English language abstract of Japanese Publication Number JP3205548 describes a sensor for detecting catecholamines. The sensor comprises an oxidising enzyme wherein the response of the electrode to catecholamines is chemically amplified whereas the electrode response to ascorbic acid is not chemically amplified.

There are a number of reports in the literature communicating the development of dopamine sensors aimed at eliminating the problems associated with ascorbic acid interference, for example, Y. F. Zhao et al. in Selective detection of dopamine in the presence of ascorbic acid and uric acid by a carbon nanotubes-ionic liquid gel modified electrode,¹ and S. B. Hocevar et al. in Carbon Nanotube Modified Microelectrode for Enhanced Voltammetric Detection of Dopamine in the Presence of Ascorbate.² The devices disclosed therein are quite complex and contain highly toxic components. In general, the methods discussed in Zhao and Hocevar (supra) function by separating the oxidation waves of ascorbic acid and dopamine, but their greatest limitation is the nature of the materials used. In particular, there is increasing evidence in the literature to show that carbon nanotubes, because of their high reactivities, may be highly toxic to biological tissues. Consequently, these materials are unlikely to be exploited in biomedical applications.

Currently carbon paste electrodes/fibres are being used as implantable dopamine sensors as these satisfy biocompatibility issues. However, these suffer interference from other species, particularly ascorbic acid. High concentrations of ascorbic acid coexist with dopamine in biological systems making the electrochemical detection of dopamine at these electrodes particularly challenging. Furthermore, while these electrodes/fibres are suitable for implantation they can only be used to carry out measurements over a 90 s period as they are easily poisoned by dopamine metabolites.

Izaoumen et al. have developed a glassy carbon electrode modified with polypyrrole and β-cyclodextrin. The resulting electrode was utilised in the selective detection of dopamine and norepinephrine in the presence of ascorbic acid.³ Temsamani et al. describe a polypyrrole sulfated 3-cyclodextrin film utilised in the detection of DOPA and metanephrine.⁴ The publication is silent to detection of DOPA and metanephrine in the presence of interferants.

The abstract of Chinese Patent Publication number 101059474 discloses an electrochemical sensor for simultaneous detection of epinephrine and ascorbic acid. The sensor comprises a Nafion layer on a glass-carbon electrode. Similarly, the abstract of Chinese Patent Publication number 1395094 describes an electrochemical sensor for dopamine comprising a glass-carbon electrode with a carbon nanotube-Nafion® film thereon. The electrode selectively detects dopamine in the presence of ascorbic acid and uric acid.

Notwithstanding the foregoing, it would still be desirable to provide a biosensor capable of reliable real-time in-situ, in-vivo measurement of catecholamines, e.g. dopamine, epinephrine, and/or norepinephrine. Further still, it would be desirable that such a biosensor be able to function in the presence of interfering molecules such as ascorbic acid and in the presence of metabolites without suffering the aforementioned problems associated with dopamine metabolite poisoning.

### SUMMARY OF THE INVENTION

The present invention relates to biosensors comprising of a conducting polymer doped with macrocyclic cages. In one aspect the present invention provides for an electrode for detecting a catecholamine comprising:

(i) a conducting or semi-conducting substrate; and

(ii) a polymer comprising polyethylenedioxythiophene on said substrate,

wherein said polymer is doped with a cyclodextrin macrocycle.

Desirably, the catecholamine is selected from dopamine, epinephrine or norepinephrine.

Desirably, the electrode of the present invention comprises a conducting substrate. As will be appreciated by a person skilled in the art the conducting substrate may comprise a metal selected from the group consisting of Pt, Ag, Au, Ru, Rh, Pd, Re, Os, Ir, Ti, Indium tin oxide (ITO) coated glass and combinations thereof. Further still, the conducting substrate may comprise a non-metallic conductor such as carbon fibres, graphite, glassy carbon, diamond, carbon paste and pyrolithic carbon electrodes, or boron doped diamond. Desirably, the conducting substrate comprises Au.

As used herein, the polymer comprising polyethylenedioxythiophene (PEDOT) is of the general structure 5, wherein n≧1.

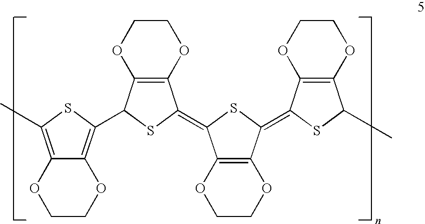

Desirably, the cyclodextrin macrocycle comprises an anionic cyclodextrin macrocycle. Further desirably, the anionic cyclodextrin macrocycle comprises an anionic α-cyclodextrin, β-cyclodextrin, γ-cyclodextrin and combinations thereof. In a preferred embodiment the anionic cyclodextrin macrocycle comprises an anionic βcyclodextrin.

It is advantageous that the cyclodextrin macrocycle of the electrode of the present invention is anionic (negatively charged). The negative charge associated with the macrocyclic cage attracts cationic (positively charged) species within the cage structure. At physiological pH metabolites known to poison prior art electrodes (for example DOPAC and homovanillic acid) are anionic species. As such the electrode construction of the present invention should not be affected by these metabolites to the same extent, as the anionic cyclodextrin should repel these anionic metabolites. The size of the cavity of the macrocyclic cage, i.e. whether α,β, or γ, may also function as a size exclusion barrier or the like, allowing complexation of molecules below a certain molecular weight only.

In a preferred embodiment the anionic cyclodextrin macrocycle comprises a sulfonated cyclodextrin macrocycle. Desirably, the sulfonated cyclodextrin macrocycle may comprise a sulfonated α-cyclodextrin, a sulfonated β-cyclodextrin, a sulfonated γ-cyclodextrin and combinations thereof. Further preferably, the anionic cyclodextrin macrocycle comprises a sulfonated β-cyclodextrin.

The electrode works by forming an inclusion complex between the macrocyclic cage immobilised in the polymer and the catecholamine. The oxidative response of the film to the catecholamine is catalytic. That is, it results in the oxidation of catecholamine occurring at a different potential than at the bare electrode (bare=gold, platinum, glassy carbon, etc.).

As used herein the term “sulfonated cyclodextrin macrocycle” refers to any cyclodextrin wherein one or more of the hydroxy groups of the glucopyranoside rings are sulfonated. Within the art the term is sometimes used interchangeably with sulfated cyclodextrin. Within this specification the terms sulfated cyclodextrin and sulfonated cyclodextrin are to be interpreted as one in the same provided the definition above is satisfied, i.e. having one or more of the hydroxy groups of the glucopyranoside rings sulfonated. For example, sulfonated β-cyclodextrin (7) is commercially available from Sigma-Alrich® as sulfated β-cyclodextrin.

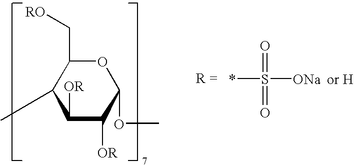

In a further aspect, the invention extends to a method of preparing an electrode for detecting a catecholamine comprising:
(i) providing a conducting or semi-conducting substrate;
(ii) providing an aqueous solution of EDOT (ethylenedioxythiophene) and an anionic cyclodextrin;
(iii) contacting said substrate and said aqueous solution; and
(iv) applying an electrical potential to provide an anionic cyclodextrin doped polyethylenedioxythiophene film on said substrate.

The anionic cyclodextrin is incorporated into the PEDOT during polymerisation as a counter ion in order to neutralise the positive charge formed on the PEDOT chain during the oxidation of the monomer. This will generally result in one cyclodextrin incorporated for every four EDOT units.

Desirably, in the method of the present invention the substrate comprises a conducting substrate. As will be appreciated by a person skilled in the art the conducting substrate may comprise a metal selected from the group consisting of Pt, Ag, Au, Ru, Rh, Pd, Re, Os, Ir, Ti, Indium tin oxide (ITO) coated glass and combinations thereof. Further still, the conducting substrate may comprise a non-metallic conductor such as carbon fibres, graphite, glassy carbon, diamond, carbon paste and pyrolithic carbon electrodes, or boron doped diamond. Desirably, the conducting substrate comprises Au.

Desirably, the cyclodextrin macrocycle comprises an anionic cyclodextrin macrocycle. Further desirably, the anionic cyclodextrin macrocycle comprises an anionic α-cyclodextrin, β-cyclodextrin, γ-cyclodextrin and combinations thereof. In one embodiment the anionic cyclodextrin macrocycle comprises an anionic β-cyclodextrin.

In a preferred embodiment of the method of the present invention the anionic cyclodextrin macrocycle comprises a sulfonated cyclodextrin macrocycle. Desirably, the sulfonated cyclodextrin macrocycle may comprise a sulfonated α-cyclodextrin, a sulfonated β-cyclodextrin, a sulfonated γ-cyclodextrin and combinations thereof. Further preferably, the anionic cyclodextrin macrocycle comprises a sulfonated β-cyclodextrin.

The invention further relates to a biosensor for detecting a catecholamine in the presence of ascorbic acid comprising:
(i) a conducting or semi-conducting substrate; and
(ii) a polymer comprising polyethylenedioxythiophene on said substrate, wherein said polymer is doped with a sulfonated β-cyclodextrin macrocycle.

As used herein ascorbic acid comprises neutral ascorbic acid and the anionic derivative ascorbate.

Desirably, the catecholamine is selected from dopamine, epinephrine or norepinephrine.

Desirably, the biosensor of the present invention comprises a conducting substrate. As will be appreciated by a person skilled in the art the conducting substrate may comprise a metal selected from the group consisting of Pt, Ag, Au, Ru, Rh, Pd, Re, Os, Ir, Ti, Indium tin oxide (ITO) coated glass and combinations thereof. Further still, the conducting substrate may comprise a non-metallic conductor such as carbon fibres, graphite, glassy carbon, diamond, carbon paste and pyrolithic carbon electrodes, or boron doped diamond. Desirably, the conducting substrate comprises Au.

The electrode and biosensor of the present invention provide for detecting catecholamines, e.g. epinephrine, dopamine, norepinephrine, in solution. As used herein the term solution comprises bodily fluids such as plasma, blood, extra-cellular fluid etc. having catecholamines dissolved therein.

The relative simplicity with which these materials can be prepared, coupled with the excellent selectivity, high biocompatibility and ease of preparation shows that these novel materials have real potential in the sensing of catecholamines and are a significant improvement on the existing technologies. The materials utilised in the electrode are highly biocompatible (PEDOT is used in tissue engineering applications and cyclodextrins are used in drug delivery) and easy to prepare (10-min preparation time).

Advantageously, the electrode and biosensor of the present invention for detecting catecholamines, e.g. epinephrine, dopamine, norepinephrine, have the potential to be miniaturised and conveniently placed in the living organism to give in-vivo data at the sub-second timescale.

Advantageously, the electrode and sensor of the present invention provide for real-time measurement of catecholamines, e.g. epinephrine, dopamine, norepinephrine, both in-vivo and in-vitro. Further still, the electrode and sensor of the present invention for detecting catecholamines, e.g. epinephrine, dopamine, norepinephrine, have the potential for in-situ monitoring.

Potential applications of the electrode and biosensor of the present invention include the evaluation of test compounds on catecholamine, e.g. epinephrine, dopamine, norepinephrine, concentrations in the brain, and the resulting neurological response.

In a further aspect of the invention, and contrary to literature reports that surfactants are necessary for polymerisation of EDOT (ethylenedioxythiophene) to occur in aqueous media, the present invention provides for a method of polymerising ethylenedioxythiophene in aqueous solution comprising:
(i) providing an aqueous solution of ethylenedioxythiophene; and
(ii) applying an electrical potential, wherein said aqueous solution does not comprise a surfactant.

### BRIEF DESCRIPTION OF THE DRAWINGS

Additional features and advantages of the present invention are described in, and will be apparent from, the detailed description of the invention and from the drawings in which:

FIG. 1 illustrates electropolymerisation of PEDOT/cyclodextrin film at a Gold electrode according to the present invention. Potential cycled from −0.5 to +1.06V vs SCE.

FIG. 2 depicts the current profile of a sulfonated β-CD doped PEDOT film on a gold electrode according to the present invention.

FIG. 3 exhibits the detection of dopamine by the electrode of the present invention at approximately 0.38V.

FIG. 4 shows detection of separate dopamine and ascorbic acid peaks utilising the electrode of the present invention.

FIG. 5 shows the voltammetric response of a sulfonated α-cyclodextrin doped PEDOT film on a gold electrode to 1×10⁻⁵ M dopamine.

FIG. 6 illustrates the response of a sulfonated α-cyclodextrin doped PEDOT film on a gold electrode to dopamine and a mixture of dopamine and ascorbic acid.

FIG. 7 is a cyclic voltammagram response of an Au electrode, coated with phosphonated β-cyclodextrin doped PEDOT, to dopamine.

FIG. 8 depicts the response of an Au electrode coated with phosphonated β-cyclodextrin doped PEDOT to solutions of dopamine and ascorbic acid.

FIG. 9 is a plot of the response of a sulfonated β-cyclodextrin doped PEDOT film on a gold electrode versus a KCl doped PEDOT film on a gold electrode to a 1×10⁻³ M dopamine solution.

FIG. 10 illustrates the voltammetric response of the PEDOT/sulfonated β-CD film on a gold electrode to epinephrine and ascorbic acid.

FIG. 11 illustrates the voltammetric response of the PEDOT/sulfonated β-CD film on a gold electrode to Norepinephrine and ascorbic acid.

### DETAILED DESCRIPTION OF THE INVENTION

### Electrode Preparation

The poor solubility of the 3,4-ethylene dioxythiophene (EDOT) monomer in aqueous solution, has led to the electropolymerisation of this monomer being predominantly performed in organic media.⁵⁵ Surfactants, such as sodium dodecyl sulphate (SDS), have been reported to improve the solubility of EDOT in aqueous and organic media.⁷ In general, it has been communicated that a critical micellar concentration (cmc) of surfactant is required in solution if polymerisation of EDOT is to occur. Cyclodextrins (CDs) have been used in place of surfactants,^{8.9} owing to the ability of cyclodextrins to form a host guest interaction with EDOT, thus increasing the solubility of the EDOT monomer in water. In the example disclosed herein, sulfonated β-cyclodextrin (β-CD) was utilised as the dopant anion necessary for film formation to occur.

The films were electropolymerised onto gold electrodes from an aqueous solution of 0.1 M ethylenedioxythiophene and 0.01 M sulfonated β-cyclodextrin, sodium salt. Polymerisation was carried out by cycling the potential between −0.5 and 1.06 V/SCE at a scan rate of 50 mV s⁻¹ for a total of three cycles.

The PEDOT/sulfonated β-cyclodextrin film properties vary depending on:
a) the EDOT:sulfonated β-CD solution concentrations (and ratio); and
b) the polymerisation technique utilised—when cyclic voltammetry is utilised the following parameters can be modified to vary the PEDOT/sulfonated β-cyclodextrin film properties;
   i) the upper (anodic) potential of the voltammetric sweep used when fabricating the polymer film. This upper potential is important for system optimisation; and
   ii) the sweep rate.

The conditions resulting in optimal catecholamine sensing comprise:
a polymerisation solution of 0.1M EDOT:0.01M sulfonated β-CD; this 10:1 ratio is important;
cyclic voltammetry (CV) is important to ensure that a homogeneous thin film is formed. Films grown using this technique exhibit enhanced dopamine signals when compared to potentiostatic growth films;
sweeping from −0.5 to +1.06 V vs SCE at a scan rate of 50 mV s-1; and
three electropolymerisation cycles to form a thin but a homogeneous film.

All scans were completed on a Solartron 1285 potentiostat. The data provided herein and in the figures were obtained using cyclic voltammetry.

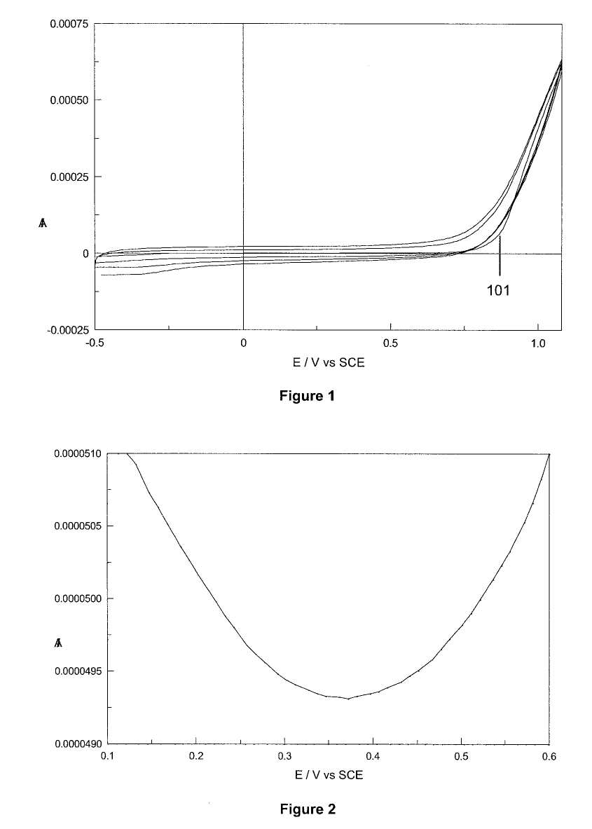

For example, in FIG. 1 we see a cyclic voltammagram of three electropolymerisation cycles of a solution of 0.1M EDOT:0.01M sulfonated β-CD. Irreversible oxidation, i.e. electropolymerisation of the monomer occurs at approximately 0.8V and leads to film formation (101). No peak in the reverse sweep direction indicates that this is essentially an irreversible process.

### Dopamine(DA) Detection

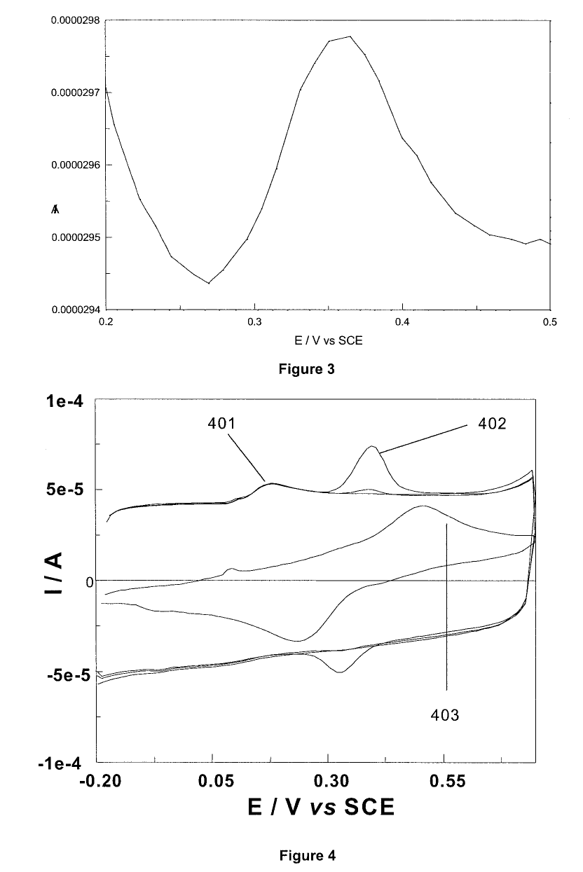

In order to detect very small concentrations of dopamine using the electrode array of the present invention, the shape of the sulfonated β-CD doped PEDOT film current profile in background electrolyte is vital. Suitable electrolyte solutions may comprise 0.1M Na₂SO₄, or 0.1 M NaCl. Desirably, the electrolyte solution comprises 0.1 M NaCl. The sulfonated β-CD doped PEDOT polymer exhibits a narrow trough or current decay in the mid-region of the oxidative sweep, as shown in FIG. 2. In FIG. 2 the trough arises at approximately 0.38V. The position of this trough can be fine tuned by varying the parameters involved in the formation of the electrode array so that the small dopamine signal ‘sits’ in it, as shown in FIG. 3. The oxidation potential of dopamine is at approximately 0.38V, as shown by the detection of a 1 μM solution of dopamine. Thus, the position of the trough should be located so that the region proximate to the oxidation potential of dopamine is not obscured by the current profile of the sulfonated β-CD doped PEDOT film.

The PEDOT/sulfonated β-CD film on a gold electrode of the present invention exhibits excellent peak separation between the peak for dopamine and that of ascorbate (AA), as shown in FIG. 4. FIG. 4 comprises a cyclic voltammagram illustrating simultaneous detection of 1×10⁻⁶, 1×10⁻⁵ & 2.5×10⁻³ M DA (402) in the presence of 1×10⁻³ M AA (401). The center trace is the bare gold response to a 1×10⁻³ M DA solution (403). The signal from dopamine is independent of the presence of ascorbic acid even at high concentrations. This selectivity also extends to other common interferants such as uric acid, DOPAC and homovanillic acid. The exclusion of these last two species is particularly advantageous as these are metabolites of dopamine which are known to poison other prior art electrodes reducing their sensitivity.

Sulfonated α-Cyclodextrin (α-CD)

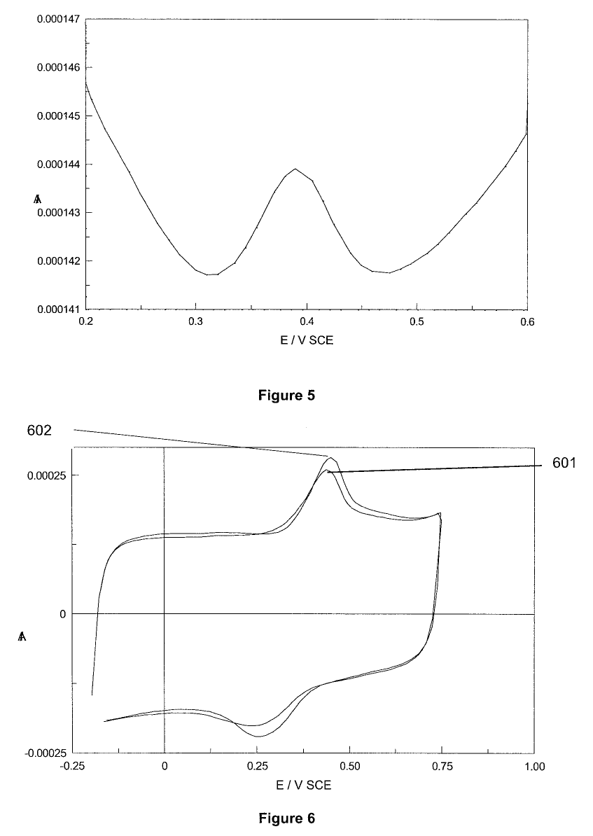

α-Cyclodextrins have a smaller cavity than β-CDs and, therefore, should exhibit different selectivity properties than those of the β form. Initial experiments have indicated that, the sulfonated α-cyclodextrin does not exhibit selectivity towards dopamine over ascorbic acid, similar to the sulfonated β-cyclodextrin discussed above. FIG. 5 shows the voltammetric response of the sulfonated α-cyclodextrin doped PEDOT film on a gold electrode to 1×10⁻⁶ M dopamine, while FIG. 6 shows the response of the sulfonated α-cyclodextrin doped PEDOT film on a gold electrode to 5×10⁻⁴ M dopamine (601) and a mixture of 5×10⁻⁴ M dopamine and 5×10⁻⁴ M ascorbic acid (602). FIG. 6 clearly illustrates that there is no separation of the ascorbic acid and dopamine peaks but a combined peak for the oxidation of both analytes is observed.

### Phosphated β-Cyclodextrin

Investigations with respect to the utility of other anionic β-CDs were performed. Results for a phosphated β-CD (Ph β-CD) are disclosed below. Akin to the sulfonated analogues, the Ph β-CD was used to dope the PEDOT film, and the resultant polymer modified electrode was used to sense dopamine.

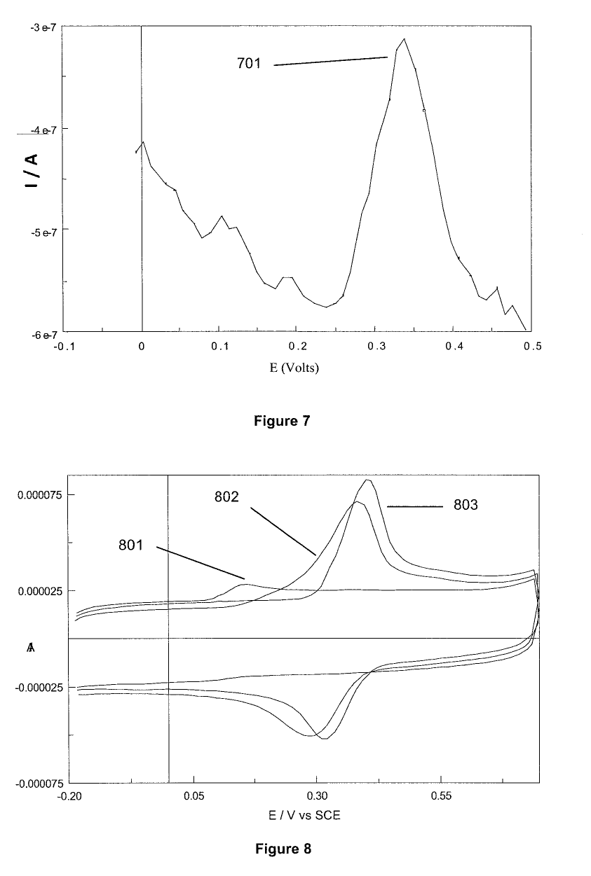

FIG. 7 depicts the cyclic voltammogram response of a Ph β-CD modified gold electrode modified to a 1×10⁻⁶ M DA solution (701). The background electrolyte contribution has been subtracted for clarity. The response is very well defined and quite substantial confirming that the film should be capable of detecting low dopamine concentrations.

FIG. 8 illustrates the voltammetric response of the Ph β-CD/PEDOT modified gold electrode to a 5×10⁻⁴ M Ascorbic Acid solution (801), a 5×10⁻⁴ M Dopamine solution (802) and a solution comprising 5×10⁻⁴ M Dopamine & 5×10⁻⁴ M Ascorbic Acid (803). The cyclic voltammogram of dopamine 802 exhibits a well defined peak at E_{P} ˜0.38V, which is ascribed to the catalytic oxidation of dopamine by the film. The film also catalyses the oxidation of ascorbic acid, with E_{P} ˜0.16V, as observed in trace 801. When the electrode was placed into the mixed solution (trace 803), we observe that the oxidative response of the electrode to both species is evident as a single well defined peak at E_{P} ˜0.4V. This peak current is of similar magnitude to the summation of peak currents for the two separate oxidations of dopamine and ascorbic acid.

Whilst the Ph β-CD/PEDOT/Au electrode was successfully used in the detection of dopamine, the selective detection of dopamine in the presence of ascorbic acid was unsuccessful as both analytes were oxidised at the same potential by the film.

### Non-Cyclodextrin Based Dopants

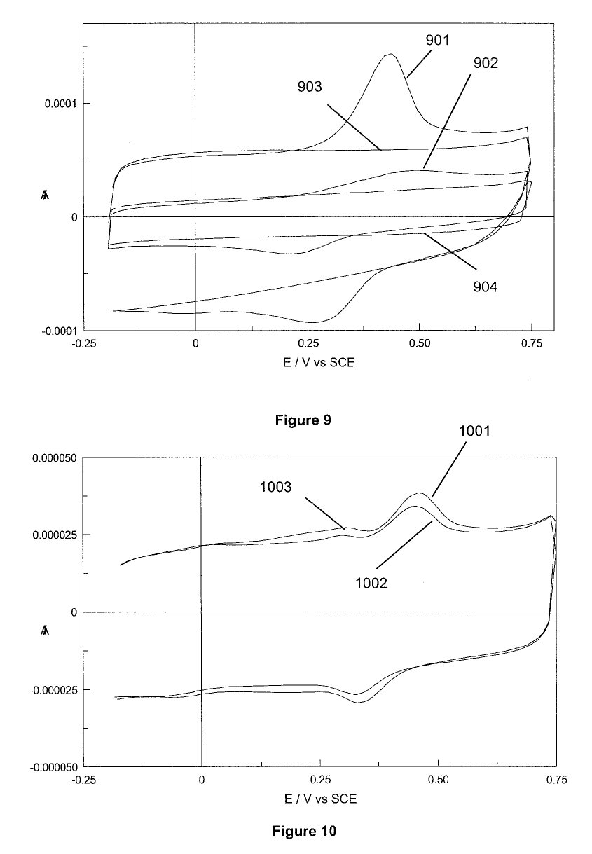

Further experiments directed to polymerising the EDOT monomer in water using KCl as the dopant were completed. Surprisingly, and contrary to literature reports that surfactants are necessary for polymerisation to occur in aqueous media, a polymer film was formed on the electrode. However, the sensing properties of this modified electrode were poor. FIG. 9 compares the response of the sulfonated β-cyclodextrin (901) PEDOT film on a gold electrode versus the KCl doped PEDOT film on a gold electrode (902), to a 1×10⁻³ M dopamine solution. The same experimental parameters were used in the fabrication of both modified electrodes and the same background electrolyte, Na₂SO₄, was used to test the sensing properties. Background scans for the sulfonated β-CD doped PEDOT film (903) and the KCl doped PEDOT film (904) can also be seen in FIG. 9.

The sulfonated β-cyclodextrin doped PEDOT film modified gold electrode (901) exhibits higher oxidation currents, thus allowing the detection of lower dopamine concentrations at the PEDOT/CD electrode than is possible at the bare electrode, and also shows a catalytic oxidative response to the Dopamine. The oxidative response of the film to dopamine is termed catalytic because it results in the oxidation of dopamine occurring at a lower potential than at the bare electrode (bare=gold, platinum or glassy carbon, etc.). Normally at the bare electrode we would see a dopamine peak at about 0.5V but with the PEDOT/CD film this potential is reduced to about 0.4V, and as such is considered to have a catalytic effect.

The KCl doped PEDOT (902) behaves as a permeable membrane through which the dopamine diffuses and is oxidised at the electrode surface. That is, the response to dopamine oxidation is not catalytic and therefore is not specific to dopamine oxidation.

### Epinephrine (EP) Detection

In FIG. 10 the voltammetric response of the PEDOT/CD film modified gold electrode to a 5×10⁻⁴ M EP solution (trace 1002) and 5×10⁻⁴ M EP in the presence of 5×10⁻⁴ M AA (trace 1001), obtained using cyclic voltammetry, is shown. There is a well defined peak due to EP oxidation at ˜0.45V. The EP oxidation peak is again evident in the presence of AA 1003.

Norepinephrine (norEP) Detection

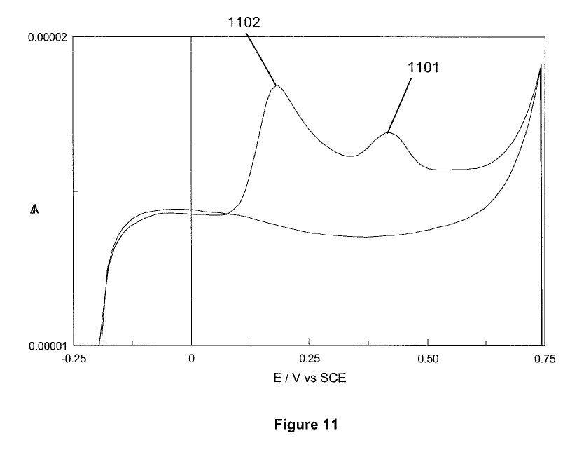

In FIG. 11 the voltammetric response of the PEDOT/CD film to a solution of 1×10⁻⁵ M norEP and 1×10⁻³ M AA obtained using cyclic voltammetry is shown. There is a well-defined peak 1101 due to norEP oxidation at ˜0.45V. The norEP oxidation peak is evident in the presence of AA peak 1102.

The words “comprises/comprising” and the words “having/including” when used herein with reference to the present invention are used to specify the presence of stated features, integers, steps or components but do not preclude the presence or addition of one or more other features, integers, steps, components or groups thereof.

It is appreciated that certain features of the invention, which are, for clarity, described in the context of separate embodiments, may also be provided in combination in a single embodiment. Conversely, various features of the invention which are, for brevity, described in the context of a single embodiment, may also be provided separately or in any suitable sub-combination.

### REFERENCES

1. Y. F. Zhao et al., Talanta, 66, 51-57 (2005).
2. S. B. Hocevar et al., Electroanalysis, 17, 417 (2005).
3. N. Izaoumen, D. Bouchta, H. Zejli, M. E l Kaoutit, K. R. Temsamani, Analytical Letters, 38 (2005), 1869-1885_{—}
4. K. R. Temsamani, H. B. Mark Jr., W. Kutner, A. M. Stalcup, J. Solid State Electrochem., 6 (2002), 391-395.
5. V. Noel, H. Randriamahazaka, C. Chevrot, J. Electroanal. Chem., 542 (2003) 33.
6. J. Bobacka, A. Lewenstam, A. Ivaska, J. Electroanal. Chem. 489 (2000) 17.
7. N. Sakmeche, J. J. Aaron, M. Fall, S. Aeiyach, M. Jouini, J. C. Lacroix, P. C. Lacaze, Chem. Commun., (1996), 2723.
8. C. Lagrost, J. C. Lacroix, S. Aeiyach, M. Jouini, K. I. Chane-Ching, P. C. Lacaze, Chem. Commun., (1998), 489.
9. V. S. Vasantha, K. L. N. Phani, J. Electroanal, Chem., 520 (2002), 79.

1. An electrode for detecting a catecholamine comprising:
(i) a conducting or semi-conducting substrate; and
(ii) a polymer comprising polyethylenedioxythiophene on said substrate, wherein said polymer is doped with a cyclodextrin macrocycle.

2. An electrode according to claim 1 wherein the catecholamine is selected from the group consisting of dopamine, epinephrine or norepinephrine.

3. An electrode according to claim 1 comprising a conducting substrate.

4. An electrode according to claim 3 wherein the conducting substrate comprises Au.

5. An electrode according to claim 1 wherein the cyclodextrin comprises an anionic cyclodextrin.

6. An electrode according to claim 5 wherein the anionic cyclodextrin comprises an anionic α-cyclodextrin, β-cyclodextrin, γ-cyclodextrin and combinations thereof.

7. An electrode according to claim 6 wherein the anionic cyclodextrin comprises an anionic β-cyclodextrin.

8. An electrode according to claim 7 wherein the anionic β-cyclodextrin comprises a sulfonated-β-cyclodextrin.

9. A biosensor for detecting a catecholamine in the presence of ascorbic acid comprising:
(i) a conducting or semi-conducting substrate; and
(ii) a polymer comprising polyethylenedioxythiophene on said substrate, wherein said polymer is doped with a sulfonated β-cyclodextrin macrocycle.

10. A biosensor according to claim 9 wherein the catecholamine is selected from the group consisting of dopamine, epinephrine or norepinephrine.

11. A biosensor according to claim 9 comprising a conducting substrate.

12. A biosensor according to claim 11 wherein the conducting substrate comprises Au.

13. A method of preparing an electrode for detecting a catecholamine comprising:
(i) providing a conducting or semi-conducting substrate;
(ii) providing an aqueous solution of ethylenedioxythiophene and an anionic cyclodextrin;
(iii) contacting said substrate and said aqueous solution; and
(iv) applying an electrical potential to provide an anionic cyclodextrin doped polyethylenedioxythiophene film on said substrate.

14. A method according to claim 13 comprising providing a conducting substrate.

15. A method according to claim 14 wherein the conducting substrate comprises Au.

16. A method according to claim 13 wherein the anionic cyclodextrin comprises an anionic α-cyclodextrin, β-cyclodextrin, γ-cyclodextrin and combinations thereof.

17. A method according to claim 16 wherein the anionic cyclodextrin comprises an anionic β-cyclodextrin.

18. A method according to claim 17 wherein the anionic β-cyclodextrin comprises a sulfonated-β-cyclodextrin.

19. A method of polymerising ethylenedioxythiophene in aqueous solution comprising:
(i) providing an aqueous solution of ethylenedioxythiophene; and
(ii) applying an electrical potential,
wherein said aqueous solution does not comprise a surfactant.
